(19) United States
(12) Patent Application Publication
(10) Pub. No.: US 2011/0004151 A1
(43) Pub. Date: Jan. 6, 2011

(54) Treatment or Therapy Apparatus

(76) Inventors: Henry Fritz Simonsen, Trustrup (DK); Michael Kiernan, South Wales (GB); Robert Marc Clement, Swansea (GB); Jan Simonsen, Struer (DK); Hanne Simonsen, Struer (DK)

Correspondence Address: GORDON & JACOBSON, P.C., 60 LONG RIDGE ROAD, SUITE 407, STAMFORD, CT, 06902, US

(21) Appl. No.: 12/667,132

(22) PCT Filed: Jun. 27, 2008
(86) PCT No.: PCT/GB2008/050516

§ 371 Date: Aug. 18, 2010

(30) Foreign Application Priority Data
Jun. 29, 2007 (GB) 0712613.9

(51) Int. Cl. A61M 35/00

(52) U.S. Cl. 604/23

(57) ABSTRACT

The apparatus is arranged to supply a flow of substantially pure oxygen to a person, and comprises a source of substantially pure oxygen gas, means for release of the substantially pure oxygen gas from the source into an ambient atmosphere; and means for cooling the substantially pure oxygen gas released from the source to a temperature below that of the ambient atmosphere. The substantially pure oxygen gas when cooled by the cooling means can be allowed to sink under ambient pressure by convection into a zone including the ambient atmosphere, the zone being beneath, and in convective communication with, the cooled substantially pure oxygen gas, whereby the cooled and sunken substantially pure oxygen gas can come into contact with the skin of a person in that zone. At least one parameter (such as temperature, oxygen concentration, sound or vibration) is monitored in the zone and operation of the release means is only permitted when the parameter is in a predetermined range.

### BACKGROUND OF THE INVENTION

1. Field of the Invention

The present invention relates to apparatus for treatment or therapy and, in particular, apparatus for oxygen treatment or therapy, and a method of cosmetic or therapeutic treatment of skin.

2. State of the Art

Oxygen is a versatile and powerful agent and is currently used in therapy in one of two ways, namely by administration under pressure and by direct administration to the respiratory passage (by inhalation).

Administration of oxygen under pressure is known as “hyperbaric oxygen therapy” and is currently used for enhancing wound healing processes and the like. The main drawbacks of hyperbaric treatment are that specialised compression apparatus is needed and that the person being treated often has to spend hours each day (sometimes overnight) in an oxygen chamber.

It is also known to dispense oxygen for inhalation either medicinally, for treatment of diseases such as emphysema or the like, or recreationally, for example, at oxygen bars to patrons who believe that inhaling pure oxygen will enhance bodily functions. Such administration requires special masks or the like to dispense the oxygen, because the oxygen is to be administered direct to the respiratory system.

### SUMMARY OF THE INVENTION

It would be advantageous to provide apparatus permitting administration of substantially pure oxygen when the person to be treated is in an atmosphere which is under ambient pressure.

The present invention accordingly provides apparatus arranged to supply a flow of substantially pure oxygen to a person, which apparatus comprises:
a source of substantially pure oxygen gas;
means for release of said substantially pure oxygen gas from said source into an ambient atmosphere;
means for cooling said substantially pure oxygen gas released from said source to a temperature below that of said ambient atmosphere; such that said substantially pure oxygen gas when cooled by said cooling means can be allowed to sink under ambient pressure by convection into a zone including said ambient atmosphere, said zone being beneath, and in convective communication with, said cooled substantially pure oxygen gas, whereby the cooled and sunken substantially pure oxygen gas can come into contact with the skin of a person in said zone; and
monitoring means for monitoring at least one parameter in the zone and for permitting operation of the release means when the parameter is in a predetermined range.

The apparatus according to the invention may include a suitable source of pure oxygen gas (such as a pressurised receptacle containing compressed or liquefied oxygen), or alternatively may include means for extracting oxygen gas from ambient air. Such extraction means may comprise one or more compressor, filters and/or purifiers.

The oxygen gas may be partially ozonised, if wished, in an amount below that which the ozone might cause deleterious effects to a user

The apparatus may include a shroud or hood adapted to be located above a user's head when the user is in a lying or recumbent position, the user when lying being either on their side or face uppermost (that is supine). Such a shroud or hood is typically spaced from (that is not in contact with) the user and typically has a concave surface arranged to face the user's head (thereby directing flow towards the user's head). In this case, the means for release of the pure oxygen gas is typically arranged to release the gas to the inner part of the concave surface such that the latter can direct the flow downwardly towards the user.

The apparatus for supply of substantially pure oxygen gas (that is, medically pure oxygen gas), is preferably subject to safety measures, and suitable usage precautions. The apparatus therefore preferably includes means for regulating the pressure and flow volume of the substantially pure oxygen gas, and also means for controlling the temperature of cooled substantially pure oxygen gas provided by the cooling means (the latter controlling the degree of cooling of the gas).

The means for release of substantially pure oxygen gas may be arranged to be manually operable (such that a user or helper may choose when to permit release of the medically pure oxygen gas) or it may be automatically operable (for example, when a door leading to the zone is opened) or it may be arranged to be triggered by a timer at predetermined times.

The ambient atmosphere into which the substantially pure oxygen gas is released using apparatus according to the invention is typically in a room such as a bedroom or clinic facility; alternatively, the ambient atmosphere may be substantially enclosed in, for example a closed chamber, a pressurised aircraft cabin or the like.

The apparatus preferably includes means for monitoring the quality of substantially pure oxygen gas being supplied. The means for monitoring preferably includes positive feedback, such that when the oxygen percentage of the ambient atmosphere in the vicinity of the user drops below a predetermined threshold, further substantially pure oxygen gas is released from the source. Such feedback may be operated via wireless control means or via wired circuitry. When wireless control is used, the latter may be arranged to send a coded signal (such as a radio frequency, optical or ultrasonic signal) to a suitable receiver.

Alternatively, the parameter being monitored may be the temperature of the zone, and the apparatus according to the invention may then be provided with a shut-off which closes the release means when the parameter is detected as being below the lower end of the abovementioned predetermined range.

In a further alternative, the parameter being monitored may, for example, be sound and/or vibration, so that if snoring is detected, the release means can be enabled and if such snoring ceases the release means is disabled.

Any or all of the above parameters may be monitored either separately or simultaneously using apparatus according to the invention.

The apparatus according to the invention may further include means for release of mood-altering chemicals (such as aromatherapy substances, which may have a relaxing or calming, or stimulating or energising effect).

The apparatus may further include means for release of therapeutic chemicals, such as anti-asthma pharmaceuticals, particularly where such chemicals are to be administered by inhalation, because the supply of the medically pure oxygen gas enhances uptake of such inhalable materials.

The apparatus may further include means for ionising the gas (for example, to impart a negative charge thereto).

The means for release of the substantially pure oxygen gas from the source into the ambient atmosphere may include at least one nozzle, such as a set of downwardly directed nozzles for application of the gas flow to a skin surface of the user and/or to a region in the vicinity of a user's face.

The apparatus according to the invention may include one or more timers arranged either to supply the substantially pure oxygen gas for a predetermined time period, and/or over one or more predetermined time intervals. It is particularly preferred that such timers are controlled to supply the substantially pure oxygen gas over a predetermined sleep period (for example, overnight, such that the flow is switched off at an expected waking time). In some embodiments, the flow of the timer is arranged to permit supply of the substantially pure oxygen gas over a plurality of spaced timed intervals.

The apparatus may further include movement sensors to detect when the user makes more than a predetermined amount of movement; these may be calibrated such that when the user wakes up and starts to move, a signal is sent to the means for release oxygen, to prevent further flow thereof.

The apparatus may further include a detector for detecting a person in the zone beneath the release means; such a detector may be arranged to send a signal to permit release of the oxygen only when a person is detected in that zone. Such a detector may, for example, be a heat sensor arranged to detect a heat source such as a person, or a pressure switch actuated when the person sits on a chair in the zone or lies on a bed in that zone.

The apparatus according to the invention is arranged such that the sinking cooled substantially pure oxygen is allowed to contact the skin of a user in the abovementioned zone; it is particularly preferred that such contact is provided with the user at rest (for example, seated in a chair, or lying down on a bed or other surface, especially when the user is in a supine position or lying on one side).

The apparatus according to the invention may be used for the treatment of a user's skin (for example, when the user is asleep); such treatment is believed to help combat the ageing effects of polluted and/or oxygen-depleted atmospheres (for example, in aircraft, in urban environments or the like). The apparatus according to the invention may thereby be used to combat wrinkles or other visibly ageing effects in the skin.

The present invention therefore further comprises a method of therapy or cosmetic treatment of a person, which method comprises cooling substantially pure oxygen gas to a temperature below the temperature of an ambient atmosphere; allowing said cooled substantially pure oxygen gas to sink under ambient pressure by convection into a zone including said ambient atmosphere, and allowing the sunken substantially pure oxygen gas to come into contact with the skin of a person in said zone.

The method according to the invention is preferably carried out using apparatus according to the invention, as has been described in the preceding description of the invention.

Preferred features of the invention will now be described further, by way of example only, with reference to the accompanying drawings.

### BRIEF DESCRIPTION OF THE DRAWINGS

FIG. 1 is a schematic diagram showing exemplary apparatus according to the invention when in use;

FIG. 2 is a schematic diagram showing a modification of the apparatus shown in FIG. 1;

FIG. 3 shows details of a hood or shroud suitable for use in apparatus according to the invention

FIG. 4 is a schematic flow chart showing a schematic view of features of exemplary apparatus according to the invention;

FIG. 5 is a schematic flow chart showing a schematic view of an alternate embodiment of the invention;

FIG. 6 is a schematic flow chart showing a schematic view of another embodiment of the invention; and

FIG. 7 is a schematic flow chart showing a schematic view of a yet another embodiment of the invention; and

### DETAILED DESCRIPTION OF THE PREFERRED EMBODIMENTS

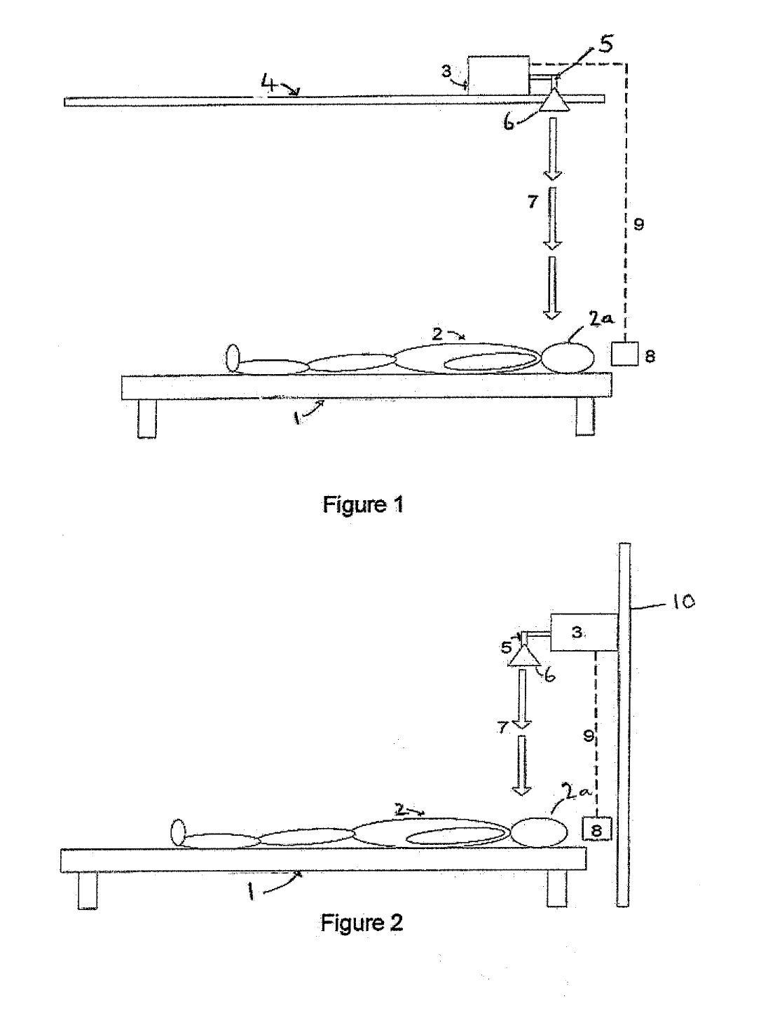

Referring firstly to FIG. 1 of the drawings, there is shown a bed 1 having a person 2 wishing to employ oxygen therapy on the bed.

Above the bed is a reservoir 3 for releasing cooled oxygen, mounted or fixed to a ceiling 4. The reservoir has an outlet connected via conduit 5 to a downwardly directed hood 6 which directs chilled pure oxygen gas from the reservoir 3 in a downwardly directed stream 7 towards the face 2a of the person 2. The cooled oxygen gas therefore sinks to come into contact with the skin on the face 2a of the person 2, causing beneficial effects to the skin such as combating ageing/wrinkling of the skin, as well as an additional benefit aiding breathing and (for example) inhibiting snoring by the person.

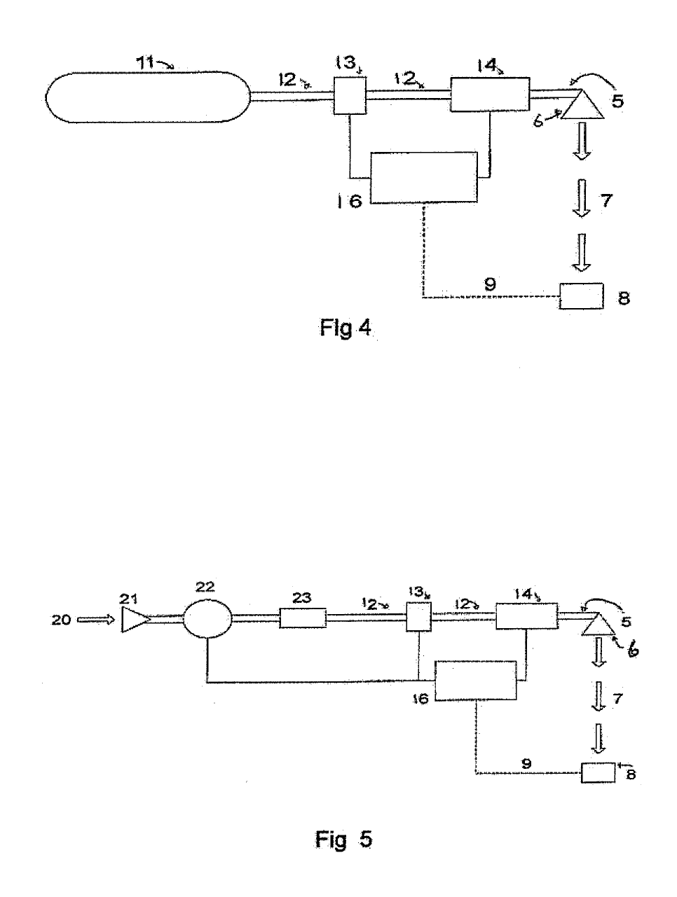

Adjacent to the face 2a is a sensor 8 for sensing conditions such as temperature or oxygen concentration in its vicinity; the sensor 8 may be connected via a data link 9 to a controller (not shown in FIG. 1—shown in more detail in FIG. 4, as described below) for controlling release of oxygen. The sensor may further include a proximity or movement sensor arranged to stop oxygen supply if the person moves away from an optimum position on the bed.

Referring to FIG. 2 of the drawings, the arrangement is similar (and like features are denoted by like reference numerals); the main difference is that the reservoir 3 is mounted to a wall 10 rather than to a ceiling.

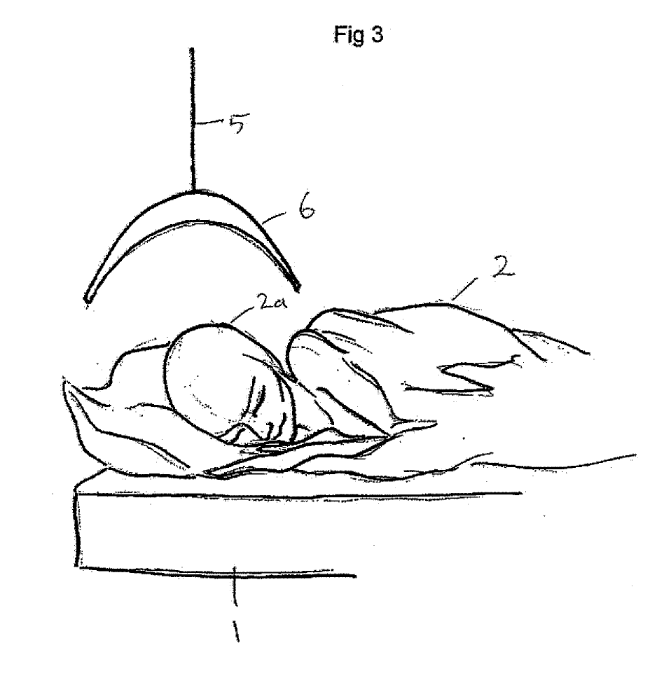

FIG. 3 (in which again like reference numerals denote like features) shows in more detail an example of the hood 6 located just above the face 2a of a person 2 on a bed 1.

FIG. 4 shows more details of a control system for use in the arrangement of FIGS. 1 and 2.

In the arrangement shown in FIG. 3, the reservoir comprises a pressurised oxygen tank 11 having an oxygen delivery tube 12 provided with a flow regulator 13. After passing the flow regulator 13, oxygen passes through a cooler unit 14 to conduit 5 before exiting through the hood 6. Regulator 13 and cooler unit 14 are both controlled by control unit 16, which receives signals from sensor 8 via data link 9.

FIG. 5 shows a modification of the arrangement of FIG. 4, but with an oxygen compressor instead of a pressurised oxygen tank. In the system of FIG. 5, like parts are again denoted by like reference numerals. The modification of FIG. 5 includes an inlet port 21 for an ambient air flow 20; the passes air to a compressor 22, from which the compressed air is passed to an oxygen concentrator 23.

As in the embodiment of FIG. 4, the regulator 13 and cooler unit 14 in the embodiment of FIG. 5 are both controlled by control unit 16, which receives signals from sensor 8 via data link. The control unit also controls compressor 22.

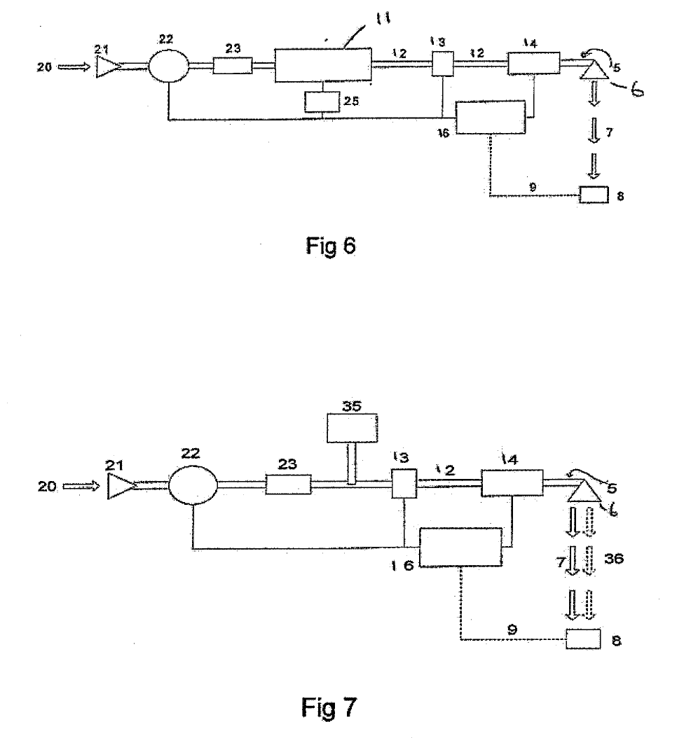

FIG. 6 shows a modification of the arrangement of FIG. 5, but with an oxygen compressor feeding to a pressurised oxygen tank. In the system of FIG. 6, like parts are again denoted by like reference numerals. The modification of FIG. 6 includes an oxygen reservoir 11 between concentrator 23 and delivery tube 12.

FIG. 7 shows a modification of the arrangement of FIG. 5, but with a medicament supply in addition. In the system of FIG. 7, like parts are again denoted by like reference numerals. The modification of FIG. 7 includes a reservoir 35 feeding medicament between concentrator 23 and flow regulator 13. This ensures a supply of medicament 36 with the oxygen flow 7.

1-13. (canceled)

14. An apparatus for supplying a flow of substantially pure oxygen to skin of a person, comprising:
(a) a source of substantially pure oxygen gas;
(b) release means for releasing said substantially pure oxygen gas from said source to an ambient atmosphere;
(c) cooling means for cooling said substantially pure oxygen gas to a temperature below ambient atmosphere such that the oxygen gas sinks from a first location above a person to a zone and can come into contact with skin of the person in the zone; and
(d) monitoring means for monitoring at least one parameter in the zone,
wherein said monitoring means includes positive feedback means for activating said release means to release substantially pure oxygen gas from said source when a measured parameter in the zone in the vicinity of the person is below a corresponding predetermined range.

15. An apparatus according to claim 14, wherein:
the source of substantially pure oxygen gas includes a pressurized receptacle containing compressed or liquefied oxygen

16. An apparatus according to claim 14, wherein:
the source of substantially pure oxygen gas includes means for extracting oxygen gas from ambient air.

17. An apparatus according to claim 14, further comprising:
a shroud or hood adapted to be positioned above the head of a person in a lying position.

18. An apparatus according to claim 14, further comprising:
regulating means for regulating the pressure and flow volume of the substantially pure oxygen gas released from the release means.

19. An apparatus according to claim 14, further comprising:
control means for controlling the temperature of cooled substantially pure oxygen gas allowed to sink from the first location.

20. An apparatus according to claim 14, wherein:
the at least one parameter monitored by the monitoring means includes oxygen percentage of the substantially pure oxygen gas released from the release means.

21. An apparatus according to claim 14, wherein:
the at least one the parameter monitored by the monitoring means includes temperature of the zone, and the apparatus includes a shut-off for shutting off release of said oxygen gas when temperature of the zone is below a predetermined temperature range.

22. An apparatus according to claim 14, further comprising:
secondary release means for releasing mood-altering chemicals or therapeutic chemicals in conjunction with said substantially pure oxygen gas.

23. An apparatus according to claim 14, further comprising:
ionizing means for ionizing the substantially pure oxygen gas.

24. An apparatus according to claim 14, further comprising:
at least one timer arranged to permit release of the substantially pure oxygen gas from the source for a predetermined time period.

25. An apparatus according to claim 14, further comprising:
at least one timer arranged to permit release of the substantially pure oxygen gas from the source over at least one predetermined time interval.

26. An apparatus according to claim 14, wherein:
the monitoring means includes at least one movement sensor for detecting movement of a person.

27. An apparatus according to claim 14, wherein:
the zone is disposed below the release means, and the monitoring means includes at least one detector for detecting a person in the zone.

28. A method of treatment of skin of a person in a zone within an ambient atmosphere at ambient pressure, the method comprising:
(a) cooling substantially pure oxygen gas released into the ambient atmosphere to a temperature below ambient temperature;
(b) allowing said cooled substantially pure oxygen gas to sink under ambient pressure by convection into a zone within said ambient atmosphere;
(c) allowing the substantially pure oxygen gas which sank in (b) to come into contact with the skin of a person in the zone;
(d) monitoring at least one parameter in the zone; and
(e) permitting release of additional cooled substantially pure oxygen gas when a parameter monitored in (d) is in a predetermined range.

29. A method of treatment of skin of a person in a zone within an ambient atmosphere at ambient pressure, the method comprising providing a source of substantially pure oxygen gas; releasing said substantially pure oxygen gas from said source into the ambient atmosphere; cooling the substantially pure oxygen gas released from the source to a temperature below ambient temperature so as to allow the cooled substantially pure oxygen gas to sink under ambient pressure by convection into the zone, the zone being beneath and in convective communication with the ambient atmosphere at and adjacent the source, whereby the cooled substantially pure oxygen gas comes into contact with the skin of the person; monitoring at least one parameter in the zone; permitting release of the substantially pure oxygen gas when a monitored parameter is in a predetermined range; and providing positive feedback when the monitored parameter is below said predetermined range in the vicinity of the person to release additional substantially pure oxygen gas from the source.
